(19) United States
(12) Patent Application Publication
(10) Pub. No.: US 2006/0105343 A1
(43) Pub. Date: May 18, 2006

(54) Methods for diagnosis and prognosis of cancer

(75) Inventors: Bruce R Zetter, Wayland, MA (US); Lloyd Hutchinson, Arlington, MA (US); Lere Bao, Maryland, MA (US)

Correspondence Address: DAVID S. RESNICK, 100 SUMMER STREET, NIXON PEABODY LLP, BOSTON, MA, 02110-2131, US

(73) Assignee: Children's Medical Center Corporation, Boston, MA (US)

(21) Appl. No.: 10/537,455

(22) PCT Filed: Jan. 7, 2004
(86) PCT No.: PCT/US04/00447

§ 371 Date: Oct. 31, 2005

Related U.S. Application Data
(60) Provisional application No. 60/438,861, filed on Jan. 9, 2003.

(51) Int. Cl. C12Q 1/68; G01N 33/574; G01N 33/00

(52) U.S. Cl. 435/6; 435/7.23; 436/86

(57) ABSTRACT

We have discovered a protein in humans, herein referred to as thymosin R16 (SEQ ID NO: 1), that is expressed in human prostate cancer tumors but not in specimens of benign prostate hyperplasma (BPH) tissues. In contrast, prostate specific antigen (PSA), the gold standard of prostate cancer diagnosis, is highly expressed in BPH tissues. Increased expression of thymosin (316 has a high correlation to disease state in a number of cancers including prostate cancer and cancers of epithelial origin. Accordingly, method of diagnosing and prognosing cancer in a patient by measuring the level of thymosin (316 in a biological test sample obtained from the patient are provided.

### RELATED APPLICATIONS

This application claims the benefit of U.S. Provisional Application No. 60/438,861, filed Jan. 9, 2003.

### STATEMENT REGARDING FEDERALLY SPONSORED RESEARCH

The work described herein was supported, in part, by National Institute of Health grant No. R01CA37393. The U.S. Government has certain rights to the invention.

### BACKGROUND OF THE INVENTION

Cancer remains a major health concern. Despite increased understanding of many aspects of cancer, the methods available for its treatment continue to have limited success. First of all, the number of cancer therapies is limited, and none provides an absolute guarantee of success. Second, there are many types of malignancies, and the success of a particular therapy for treating one type of cancer does not mean that it will be broadly applicable to other types. Third, many cancer treatments are associated with toxic side effects. Most treatments rely on an approach that involves killing off rapidly growing cells; however, these treatments are not specific to cancer cells and can adversely affect any dividing healthy cells. Fourth, assessing molecular changes associated with cancerous cells remains difficult. Given these limitations in the current arsenal of anti-cancer treatments, how can the best therapy for a given patient be designed? The ability to detect a malignancy as early as possible, and assess its severity, is extremely helpful in designing an effective therapeutic approach. Thus, methods for detecting the presence of malignant cells and understanding their disease state are desirable, and will contribute to our ability to tailor cancer treatment to a patient's disease.

While different forms of cancer have different properties, one factor which many cancers share is the ability to metastasize. Until such time as metastasis occurs, a tumor, although it may be malignant, is confined to one area of the body. This may cause discomfort and/or pain, or even lead to more serious problems including death, but if it can be located, it may be surgically removed and, if done with adequate care, be treatable. However, once metastasis sets in, cancerous cells have invaded the body and while surgical resection may remove the parent tumor, this does not address other tumors. Only chemotherapy, or some particular form of targeting therapy, then stands any chance of success.

The process of tumor metastasis is a multistage event involving local invasion and destruction of the intracellular matrix, intravasation into blood vessels, lymphatics or other channels of transport, survival in the circulation, extravasation out of the vessels in the secondary site(s), and growth in the new location(s) (Fidler, et al., Adv. Cancer Res. 28, 149-250 (1978), Liotta, et al., Cancer Treatment Res. 40, 223-238 (1988), Nicolson, Biochim. Biophy. Acta 948, 175-224 (1988) and Zetter, N. Eng. J. Med. 322, 605-612 (1990)). Success in establishing metastatic deposits requires tumor cells to be able to accomplish these steps sequentially. Common to many steps of the metastatic process is a requirement for motility. The enhanced movement of malignant tumor cells is a major contributor to the progression of the disease toward metastasis. Increased cell motility has been associated with enhanced metastatic potential in animal as well as human tumors (Hosaka, et al., Gann 69, 273-276 (1978) and Haemmerlin, et al., Int. J. Cancer 27, 603-610 (1981)).

Tumor angiogenesis is essential for both primary tumor expansion and metastatic tumor spread (Blood et al., Biochim. Biophy4. Acta 1032:89-118 (1990)). Angiogenesis is a fundamental process by which new blood vessels are formed. Progressive tumor growth necessitates the continuous induction of new capillary blood vessels which converge upon the tumor. In addition, the presence of blood vessels within a tumor provides a ready route for malignant cells to enter the blood stream and initiate metastasis. Thus, malignancy is a systemic disease in which interactions between the neoplastic cells and their environment play a crucial role during evolution of the pathological process (Fidler, I. J., Cancer Metastasis Rev. 5:29-49 (1986)).

Identifying factors that are associated with tumor progression, particularly metastasis and angiogenesis, is clearly a prerequisite not only for a full understanding of cancer, but also for the development of rational new anti-cancer therapies. In addition to using such factors for diagnosis and prognosis, these factors represent important targets for identifying novel anti-cancer compounds, and are useful for identifying new modes of treatment, such as inhibition of metastasis. One difficulty, however, is that the genes characteristic of cancerous cells are very often host genes being abnormally expressed. For example, a protein marker for a given cancer, while expressed in high levels in connection with that cancer, may also be expressed elsewhere throughout the body, albeit at reduced levels. Thus, some care is required in determining whether the expression of any single gene in a given cancer is a meaningful marker for the progression of the disease.

Prostatic carcinoma is the most prevalent form of cancer in males and the second leading cause of cancer death among older males (Boring, et al., Cancer J. Clinicians, 7-26 (1994)). Clinically, radical prostatectomy offers a patient with locally contained disease an excellent chance for cure. If diagnosed after metastases are established, however, prostate cancer is a fatal disease, for which there is no effective treatment that significantly increases survival. The recent development of the prostate specific antigen (PSA) test has dramatically improved diagnosis, allowing earlier detection of prostate cancer and thus earlier treatment (Catalona, et al., J. Urol., 151, 1283-1290 (1994)). Unfortunately, the PSA test does not predict which tumors may progress to the metastatic stage (Cookson, et al., J. Urology 154, 1070-1073 (1995) and Aspinall, et al., J. Urology 154, 622-628 (1995)). In addition, up to 75% of men who test positive for serum PSA do not have prostate cancer (Caplan & Kratz, Am. J. Clin. Pathol., 117:S104-108 (2002); and Woolf, Int. J. Technol. Assess Health Care, 17(3):275-304 (2001). Such false positives lead to unenecessary medical procedures, and needless anxiety for a large number of men each year. Thus, there is a need in the art for additional biomarkers which can, alone or in combination with PSA or other biomarkers, increase the specificity and sensitivity of prostate cancer diagnosis. Additionally, the treatment and diagnosis of a variety of cancers would be significantly improved by methods for earlier detection, as well as by methods to assess the severity and metastatic potential of an individual's cancer.

### SUMMARY OF THE INVENTION

We have discovered a protein in humans, herein referred to as thymosin β16 (SEQ ID NO:1), that is expressed in human prostate cancer tumors but not in specimens of benign prostate hyperplasma (BPH) tissues. In contrast, prostate specific antigen (PSA), the gold standard of prostate cancer diagnosis, is highly expressed in BPH tissues.

These results indicate that increased expression of thymosin β16 has a high correlation to disease state in cancers including prostate cancer, lung carcinoma, breast carcinoma, thyroid carcinoma, brain cancers other than neuroblastoma (cerebellum, medulloblastoma, astrocytoma, ependymoma, glioblastoma), pancreatic carcinoma, ovarian carcinoma, uterine cancer, eye cancer (retinoblastoma), muscle (rhabdosarcoma), lymphoma, stomach cancer, liver cancer, colon cancer, kidney cancer, and is particularly associated with metastatic cancers. Preferably the cancer is of epithelial origin. As used herein the cancer is a cancer other than neuroblastoma. Preferably cancers other than brain cancer. Accordingly, assaying for enhanced levels of transcript or gene product can be used not only in a diagnostic manner, but also in a prognostic manner for particular cancers. Additionally, thymosin β16 can be used alone or in conjunction with other cancer markers, e.g., PSA and thymosin β15, in the diagnosis and prognosis of cancer. For example, PSA is a widely used diagnostic for prostate cancer, however detection of PSA leads to many false positives as well as false negatives. Monitoring the presence of thymosin β16 along with levels of PSA increases the specificity and sensitivity of diagnosis of prostate cancer. In addition, as levels of of thymosin β16 are elevated in a variety of cancers, using methods for detection of thymosin β16 in conduction with specific cancer biomarkers increases the specificity and sensitivity of diagnosis using the specific markers.

The present invention provides a method of diagnosing cancer in a patient, especially cancers of epithelial origin, such as prostate cancer. The method comprises measuring the level of thymosin β16 in a biological test sample obtained from the patient and comparing the observed level of thymosin β16 with the level of thymosin β16 present in a normal control sample of the same type. Higher levels of thymosin β16 in the test sample, as compared to the normal control sample, is indicative of cancer.

The term “normal control sample” refers to a biological sample obtained from a “normal” or “healthy” individual that does not have cancer. A normal control sample can also be a sample that contains the same concentration of thymosin β16 normally present in a biological sample obtained from a healthy individual that does not have cancer.

The term “test sample” refers to a biological sample obtained from a patient suspected of having cancer.

Biological samples include, for example, blood, tissue, serum, stool, urine, sputum, cerebrospinal fluid, nipple aspirate, and supernatant from cell lysate.

In another aspect, the present invention provides a method for prognostic evaluation of a patient suspected of having, or having, a cancer. The method comprises the steps of i) measuring the level of thymosin β16 present in a test sample obtained from the patient, ii) comparing the level of thymosin β16 in the test sample to a range of thymosin β16 known to be present in biological samples of the same type, which are obtained from healthy patients that do not have cancer, and iii) evaluating the prognosis of the patient based on the comparison, where a high level of thymosin β16 in the test sample indicates an aggressive form of cancer (e.g. metastatic or invasive) and therefore a poor prognosis.

Thymosin β16 mRNA or protein may be measured to obtain thymosin β16 levels.

The present invention also contemplates the assessment of the level of thymosin β16 present in multiple test samples obtained from the same patient, where a progressive increase in the amount of thymosin β16 over time indicates an increased aggressiveness (e.g. metastatic potential) of the cancer/tumor.

In the methods of the present invention, levels of thymosin β16 can be ascertained by measuring the protein directly or indirectly by measuring mRNA transcript encoding thymosin β16. mRNA levels can be measured, for example, using Northern blot analysis or an RNA dependent polymerase chain reaction, e.g., reverse transcriptase PCR (RT-PCR). DNA chip technology may also be used to measure mRNA levels.

The present invention also provides a method for measuring thymosin β16 levels which comprises the steps of:

contacting a biological specimen with an antibody or antibody fragment which selectively binds thymosin β16, and

detecting whether said antibody or said antibody fragment is bound by said sample and thereby measuring the levels of thymosin β16.

In still another embodiment of this invention, the protein can serve as a target for agents that disrupt its function. Such agents include compounds or antibodies that bind to thymosin Pβ16 such that its function is inhibited. For example, one can add an effective amount of a compound that binds to thymosin β16 to disrupt function and thus inhibit metastasis. In another embodiment, one can use thymosin β16 expressing cells in an assay to discover compounds that bind to or otherwise interact with this protein in order to discover compounds that can be used to inhibit metastasis.

In a further embodiment of the invention, thymosin β16 or an immunogenic polypeptide thereof (or DNA encoding the protein or polypeptide) may be used in a pharmaceutical composition or vaccine to treat cancer or to inhibit the development of cancer.

In accordance with yet another aspect of the present invention, there are provided isolated antibodies or antibody fragments which selectively bind human thymosin β16. The antibody fragments include, for example, Fab, Fab′, F(ab′)2 or Fv fragments. The antibody may be a single chain antibody, a humanized antibody or a chimeric antibody.

The term “isolated” means that the material is removed from its original environment (e.g., the natural environment if it is naturally occurring). For example, naturally-occurring polynucleotides or polypeptides present in a living animal are not isolated, but the same polynucleotides or DNA or polypeptides, separated from some or all of the coexisting materials in the natural system, are isolated. Such polynucleotides could be part of a vector and/or such polynucleotides or polypeptides could be part of a composition, and still be isolated in that such vector or composition is not part of its natural environment.

The present invention further provides a method of treating a neoplastic cell expressing human thymosin β16 by administering to the cell an effective amount of a compound which suppresses the activity or production of the human thymosin β16. Preferably, the compound interferes with the expression of the human thymosin β16 gene. Such compounds include, for example, antisense oligonucleotides, si RNAs, ribozymes, RNAi, antibodies, including single chain antibodies and fragments thereof and aptamers.

As used herein, “thymosin β16” means a protein having the amino acid sequence of SEQ ID NO:1.

As used herein, the term “unique fragment” refers to a portion of the thymosin β16 nucleotide sequence or thymosin β16 protein that contains sequences (either nucleotides or amino acid residues) present in thymosin β16 (SEQ ID NOs: 1, 2, or 3) but not in other members of the thymosin family. This can be determined when the hybridization profile of that fragment under stringent conditions is such that it does not hybridize to other members,of the thymosin family. Such fragments can also be ascertained by sequence comparison. Preferably, the unique nucleotide sequence fragment is at least 10 nucleotides in length, more preferably at least 20 nucleotides in length, most preferably at least 30 nucleotides in length. Preferably, the unique polypeptide sequence fragment is 4 to 20 amino acids in length, more preferably, 6 to 15 amino acids, most preferably, 6 to. 10 amino acids.

Other aspects of the invention are disclosed infra.

### BRIEF DESCRIPTION OF THE DRAWINGS

The accompanying drawings, which are incorporated in and constitute a part of this specification, illustrate embodiments of the invention and, together with the description, serve to explain the objects, advantages, and principles of the invention.

FIG. 1 shows a western blot showing the level of endogenous β-thymosin proteins (Tβ4, Tβ15, Tβ16) in the LnCaP human prostate cancer cell line, originally derived from a lymph node metastasis, and in low or high metastatic variants of the Dunning rat prostatic carcinoma cell line.

FIG. 2 shows a summary of the immunohistochemical staining data for Tβ16 in human prostate specimens. Results for 10 specimens of benign prostatic hyperplasia and 51 prostatic carcinomas with Gleason scores ranging from 4 to 10 are shown. Each symbol represents the speciment from a different individual. Positive: samples with homogeneous staining of more than 75% of tumor cells. Parital: samples with heterogeneous staining of 10-75% of tumor cells. Negative: less than 10% of cells have a positive signal.

FIG. 3 shows Northern blot analysis of β-thymosin RNA expression in low and high metastatic cell lines: carcinoma cell lines from pancreas; BxPC3 (Bx), prostate; Dunning Rat (Dunning), LnCaP, DU145 (DU), PC3M, and breast; MDA-MB 231 and 435 variants. Total RNA from parental (P) cell lines, or subclones that exhibit low (L) or high (H) metastatic properties in the SCID mouse model, were analyzed with β-thymosin specific or β-actin 32P labeled DNA probes.

FIGS. 4A-C shows Tβ16 mRNA expression in normal human adult and fetal tissues. FIG. 4A: Northern dot blot analysis of a Clontech RNA Master Blot (#7770-1) containing mRNA from human adult and fetal tissues using a Tβ16 specific ³²P labeled DNA probe. FIG. 4B: shows that type and position of polyA+ RNAs and controls dotted on nylon membrane in the Clontech RNA Master Blot (#7770-1). The range of Tβ16 signal intensity for each tissue on the northern blot in FIG. 4A is also indicated (+ to +++++). FIG. 4C: shows Northern dot blot analysis of a Clontech RNA Master Blot (#7770-1) containing mRNA from human adult and fetal tissues using a β-actin specific ³²P labeled DNA probe.

FIG. 5 shows a table of a variety of cancers found to have increased expression of Tβ16. Increased xpression of Tβ16 as compared to non-cancerous tissue was monitored by (a) Antibody staining (b) Express Sequence Tag analysis, (c) Serial Analysis of Gene Expression (SAGE) (d) Gene Chip Array (e) or Northern Blot analysis.

FIG. 6 shows Northern Blot analysis of Tβ16 detected in various normal human tissues.

### DETAILED DESCRIPTION OF THE INVENTION

We have discovered that thymosin β16 is associated with prostate cancer but not BPH. Thus, expression of thymosin β16 is useful as a diagnostic or prognostic indicator of prostate cancer, and other cancers, particularly cancers of epithelial origin. In addition, the protein provides a target for cancer treatment.

As used herein, “cancers of epithelial origin” refers to cancers that arise from epithelial cells which include, but are not limited to, breast cancer, basal cell carcinoma, adenocarcinoma, gastrointestinal cancer, lip cancer, mouth cancer, esophageal cancer, small bowel cancer and stomach cancer, colon cancer, liver cancer, bladder cancer, pancreas cancer, ovary cancer, cervical cancer, lung cancer, breast cancer and skin cancer, such as squamus cell and basal cell cancers, prostate cancer, renal cell carcinoma, and other known cancers that effect epithelial cells throughout the body.

The term “aggressive” or “invasive” with respect to cancer refers to the proclivity of a tumor for expanding beyond its boundaries into adjacent tissue (Darnell, J. (1990), Molecular Cell Biology, Third Ed., W. H. Freeman, N.Y.). Invasive cancer can be contrasted with organ-confined cancer wherein the tumor is confined to a particular organ. The invasive property of a tumor is often accompanied by the elaboration of proteolytic enzymes, such as collagenases, that degrade matrix material and basement membrane material to enable the tumor to expand beyond the confines of the capsule, and beyond confines of the particular tissue in which that tumor is located.

The term “metastasis”, as used herein, refers to the condition of spread of cancer from the organ of origin to additional distal sites in the patient. The process of tumor metastasis is a multistage event involving local invasion and destruction of intercellular matrix, intravasation into blood vessels, lymphatics or other channels of transport, survival in the circulation, extravasation out of the vessels in the secondary site and growth in the new location (Fidler, et al., Adv. Cancer Res. 28, 149-250 (1978), Liotta, et al., Cancer Treatment Res. 40, 223-238 (1988), Nicolson, Biochim. Biophy. Acta 948, 175-224 (1988) and Zetter, N. Eng. J. Med. 322, 605-612 (1990)). Increased malignant cell motility has been associated with enhanced metastatic potential in animal as well as human tumors (Hosaka, et al., Gann 69, 273-276 (1978) and Haemmerlin, et al., Int. J. Cancer 27, 603-610 (1981)).

As used herein, a “biological sample” refers to a sample of biological material obtained from a patient, preferably a human patient, including a tissue, a tissue sample, a cell sample (e.g., a tissue biopsy, such as, an aspiration biopsy, a brush biopsy, a surface biopsy, a needle biopsy, a punch biopsy, an excision biopsy, an open biopsy, an incision biopsy or an endoscopic biopsy), and a tumor sample. Biological samples can also be biological fluid samples e.g., blood, urine, nipple aspirates.

The present invention also encompasses the use of isolates of a biological sample in the methods of the invention. As used herein, an “isolate” of a biological sample (e.g., an isolate of a tissue or tumor sample) refers to a material or composition (e.g., a biological material or composition) which has been separated, derived, extracted, purified or isolated from the sample and preferably is substantially free of undesirable compositions and/or impurities or contaminants associated with the biological sample.

As used herein, a “tissue sample” refers to a portion, piece, part, segment, or fraction of a tissue which is obtained or removed from an intact tissue of a subject, preferably a human subject. A preferred tissue sample is mammary tissue.

As used herein, a “tumor sample” refers to a portion, piece, part, segment, or fraction of a tumor, for example, a tumor which is obtained or removed from a subject (e.g., removed or extracted from a tissue of a subject), preferably a human subject.

As used herein, a “primary tumor” is a tumor appearing at a first site within the subject and can be distinguished from a “metastatic tumor” which appears in the body of the subject at a remote site from the primary tumor.

The present invention is directed to methods for diagnosis of cancer in a patient. The methods involve measuring levels of thymosin β16 in a test sample obtained from a patient, suspected of having cancer, and comparing the observed levels to levels of thymosin β16 found in a normal control sample, for example a sample obtained from a patient that does not have cancer. Levels of thymosin β16 higher than levels that are observed in the normal control indicate the presence of cancer. The levels of thymosin β16 can be represented by arbritary units, for example as units obtained from a densitometer, luminometer, or an ELISA plate reader.

As used herein, “a higher level of thymosin β16 in the test sample as compared to the level in the normal control sample” refers to an amount of thymosin β16 that is greater than an amount of thymosin β16 present in a normal control sample. The term “higher level” refers to a level that is statistically significant or significantly above levels found in th normal control sample. Preferably, the “higher level” is at least 2 fold greater.

The term “statistically significant” or “significantly” refers to statistical significance and generally means a two standard deviation (2SD) above normal, or higher, concentration of the marker.

As used herein, “a high level” of thymosin β16 refers to amounts of thymosin β16 that are at least 3 fold greater than the amounts of thymosin β16 present in normal control samples, preferably 5 fold to 6 fold greater.

For purposes of comparison, the test sample and normal control sample are of the same type, that is, obtained from the same biological source. The normal control sample can also be a standard sample that contains the same concentration of thymosin β16 that is normally found in a biological sample of the same type and that is obtained from a healthy individual. For example, there can be a standard normal control sample for the amounts of thymosin β16 normally found in biological samples such as urine, blood, cerebral spinal fluid, or tissue.

Additionally, disease progression can be assessed by following thymosin β16 levels in individual patients over time. Cancers include, for example, prostate cancer, lung carcinoma, breast carcinoma, thyroid carcinoma, brain cancers (cerebellum, medulloblastoma, astrocytoma, ependymoma, glioblastoma), pancreatic carcinoma, ovarian carcinoma, eye cancer (retinoblastoma), muscle (rhabdosarcoma), lymphoma, stomach cancer, liver cancer, colon cancer, kidney cancer.

The present invention further provides for methods of prognostic evaluation of a patient suspected of having, or having, cancer. The method comprises measuring the level of thymosin β16 present in a test biological sample obtained from a patient and comparing the observed level with a range of thymosin β16 levels normally found in biological samples (of the same type) of healthy individuals. A high level for example, is indicative of a greater potential for metastatic activity and corresponds to a poor prognosis, while lower levels indicate that the tumor is less aggressive and correspond to a better prognosis.

This information can be used by the physician in determining the most effective course of treatment. A course of treatment refers to the therapeutic measures taken for a patient after diagnosis or after treatment for cancer. For example, a determination of the likelihood for cancer recurrence, spread, or patient survival, can assist in determining whether a more conservative or more radical approach to therapy should be taken, or whether treatment modalities should be combined. For example, when cancer recurrence is likely, it can be advantageous to precede or follow surgical treatment with chemotherapy, radiation, immunotherapy, biological modifier therapy, gene therapy, vaccines, and the like, or adjust the span of time during which the patient is treated.

Changes in a patient's condition can be monitored using the methods of the present invention by comparing changes in thymosin β16 expression levels in the tumor in that subject over time.

Biological specimens include, for example, blood, tissue, serum, stool, urine, sputum, nipple aspirates, cerebrospinal fluid and supernatant from cell lysate. Preferably, one uses tissue specimens, serum or urine. The determination of, and comparison of, thymosin β16 levels is by standard modes of analysis based upon the present disclosure.

The methods of the invention can also be practiced, for example, by selecting a combination of thymosin β16 and one or more biomarkers for which increased or decreased expression correlates with cancer, such as any of thymosin β15 (See for example PCT publication WO 97/48805), thymosin β4, thymosin β10, cIAP2, Apafl, Bcl-2, Smac, or another known or standard biomarker for cancer. The selected biomarker can be a general diagnostic or prognostic marker useful for multiple types of cancer, such as CA 125, CEA or LDH, or can be a cancer-specific diagnostic or prognostic marker, such as a colon cancer marker (for example, sialosyl-TnCEA, CA19-9, or LASA), breast cancer marker (for example, CA 15-2. Her-2/neu and CA 27.29), ovarian cancer marker (for example, CA72-4), lung cancer (for example, neuron-specific enolase (NSE) and tissue polypeptide antigen (TPA)), prostate cancer (for example, PSA, prostate-specific membrane antigen and prostatic acid phosphatase), melanoma (for example, S-100 and TA-90), as well as other biomarkers specific for other types of cancer. Those skilled in the art will be able to select useful diagnostic or prognostic markers for detection in combination with thymosin β16. Similarly, three or more, four or more or five or more or a multitude of biomarkers can be used together for determining a diagnosis or prognosis of a patient.

Thymosin β16 Detection Techniques

The present invention features agents which are capable of detecting Thymosin β16 polypeptide or mRNA such that the presence of Thymosin β16 is detected and/or quantitated. As defined herein, an “agent” refers to a substance which is cabable of identifying or detecting Thymosin Pβ16 in a biological sample (e.g., identifies or detects Thymosin β16 mRNA, Thymosin β16 DNA, Thymosin Pβ16 protein). In one embodiment, the agent is a labeled or labelable antibody which specifically binds to Thymosin β16 polypeptide. As used herein, the phrase“labeled or labelable” refers to the attaching or including of a label (e.g., a marker or indicator) or ability to attach or include include a label (e.g., a marker or indicator). Markers or indicators include, but are not limited to, for example, radioactive molecules, colorimetric molecules, and enzymatic molecules which produce detectable changes in a substrate.

In one embodiment the agent is an antibody which specifically binds to all or a portion of a Thymosin β16 protein. As used herein, the phrase “specifically binds” refers to binding of, for example, an antibody to an epitope or antigen or antigenic determinant in such a manner that binding can be displaced or competed with a second preparation of identical or similar epitope, antigen or antigenic determinant. In an exemplary embodiment, the agent is an antibody which specifically binds to all or a portion of the human Thymosin β16 protein. The term “specifically binds” means that the antibody binds only to Thymosin β16 protein and not other members of the thymosin family, such as Thymosin β10, Thymosin β4, or Thymosin β15.

In yet another embodiment the agent is a labeled or labelable nucleic acid probe capable of hybridizing to Thymosin β16 mRNA. For example, the agent can be an oligonucleotide primer for the polymerase chain reaction which flank or lie within the nucleotide sequence encoding human Thymosin β16. In a preferred embodiment, the biological sample being tested is an isolate, for example, RNA. In yet another embodiment, the isolate (e.g., the RNA) is subjected to an amplification process which results in amplification of Thymosin β16 nucleic acid. As defined herein, an “amplification process” is designed to strengthen, increase, or augment a molecule within the isolate. For example, where the isolate is mRNA, an amplification process such as RT-PCR can be utilized to amplify the mRNA, such that a signal is detectable or detection is enhanced. Such an amplification process is beneficial particularly when the biological, tissue, or tumor sample is of a small size or volume.

Standard detection techniques well known in the art for detecting RNA, DNA, proteins and peptides can readily be applied to detect thymosin β16 or its transcript to diagnose cancer, especially metastatic cancer, or to confirm that a primary tumor has, or has not, reached a particular metastatic phase. Such techniques may include detection with nucleotide probes or may comprise detection of the protein by, for example, antibodies or their equivalent. Preferably, the nucleotide probes hybridize to the sequence shown in SEQ ID Nos:2 or 3 for thymosin β16.

Thymosin β16 Nucleic Acid Probes

Types of probe include cDNA, riboprobes, synthetic oligonucleotides and genomic probes. The type of probe used will generally be dictated by the particular situation, such as riboprobes for in situ hybridization, and cDNA for Northern blotting, for example. Most preferably, the probe is directed to nucleotide regions unique to the protein. Detection of the thymosin β16 encoding gene, per se, will be useful in screening for mutations associated with enhanced expression. Other forms of assays to detect targets more readily associated with levels of expression—transcripts and other expression products—will generally be useful as well. The probes may be as short as is required to differentially recognize thymosin β16 mRNA transcripts, and may be as short as, for example, 15 bases; however, probes of at least 17 bases, more preferably 18 bases and still more preferably 20 bases are preferred.

A probe may also be reverse-engineered by one skilled in the art from the amino acid sequence of SEQ ID NO: 1. However use of such probes may be more limited than the native DNA sequence, as it will be appreciated that any one given reverse-engineered sequence will not necessarily hybridize well, or at all, with any given complementary sequence reverse-engineered from the same peptide, owing to the degeneracy of the genetic code. This is a factor common in the calculations of those skilled in the art, and the degeneracy of any given sequence is frequently so broad as to yield a large number of probes for any one sequence.

The form of labeling of the probes may be any that is appropriate, such as the use of radioisotopes, for example, ³²P and ³⁵S. Labeling with radioisotopes may be achieved, whether the probe is synthesized chemically or biologically, by the use of suitably labeled bases.

Thymosin β16 RNA Detection Techniques

Detection of RNA transcripts may be achieved by Northern blotting, for example, wherein a preparation of RNA is run on a denaturing agarose gel, and transferred to a suitable support, such as activated cellulose, nitrocellulose or glass or nylon membranes. Radiolabeled cDNA or RNA is then hybridized to the preparation, washed and analyzed by autoradiography.

Detection of RNA transcripts can further be accomplished using known amplification methods. For example, it is within the scope of the present invention to reverse transcribe mRNA into cDNA followed by polymerase chain reaction (RT-PCR); or, to use a single enzyme for both steps as described in U.S. Pat. No. 5,322,770, or reverse transcribe mRNA into cDNA followed by symmetric gap ligase chain reaction (RT-AGLCR) as described by R. L. Marshall, et al., PCR Methods and Applications 4: 80-84 (1994).

Other known amplification methods which can be utilized herein include but are not limited to the so-called “NASBA” or “3SR” technique described in PNAS USA 87: 1874-1878 (1990) and also described in Nature 350 (No. 6313): 91-92 (1991); Q-beta amplification as described in published European Patent Application (EPA) No. 4544610; strand displacement amplification (as described in G. T. Walker et al., Clin. Chem. 42: 9-13 (1996) and European Patent Application No. 684315; and target mediated amplification, as described by PCT Publication WO9322461.

In situ hybridization visualization may also be employed, wherein a radioactively labeled antisense RNA probe is hybridized with a thin section of a biopsy sample, washed, cleaved with RNase and exposed to a sensitive emulsion for autoradiography. The samples may be stained with haematoxylon to demonstrate the histological composition of the sample, and dark field imaging with a suitable light filter shows the developed emulsion. Non-radioactive labels such as digoxigenin may also be used.

Alternatively, mRNA expression can be detected on a DNA array, chip or a microarray. Oligonucleotides corresponding to the thymosin β16 are immobilized on a chip which is then hybridized with labeled nucleic acids of a test sample obtained from a patient. Positive hybridization signal is obtained with the sample containing thymosin β16 transcripts. Methods of preparing DNA arrays and their use are well known in the art. (See, for example U.S. Pat. Nos: 6,618,6796; 6,379,897; 6,664,377; 6,451,536; 548,257; U.S. 20030157485 and Schena et al. 1995 Science 20:467-470; Gerhold et al. 1999 Trends in Biochem. Sci. 24, 168-173; and Lennon et al. 2000 Drug discovery Today 5: 59-65, which are herein incorporated by reference in their entirety). Serial Analysis of Gene Expression (SAGE) can also be performed (See for example U.S. Patent Application 20030215858).

To monitor mRNA levels, for example, mRNA is extracted from the biological sample to be tested, reverse transcribed, and fluorescent-labeled cDNA probes are generated. The microarrays capable of hybridizing to thymosin β16 cDNA are then probed with the labeled cDNA probes, the slides scanned and fluorescence intensity measured. This intensity correlates with the hybridization intensity and expression levels.

Thymosin β16 Antibodies

Antibodies may be raised against either a peptide of thymosin β16 or the whole molecule. For example, a peptide may be presented together with a carrier protein, such as an KLH, to an animal system or, if it is long enough, say 25 amino acid residues, without a carrier. Antibodies can also be raised against homologs or orthologs of Thymosin β16. Modified Thymosin β16 proteins may also be used, for example chemically modified proteins (e.g. methylation, acetylation, or others), fusion proteins, or mutants. All that is required is that the antibody produced specifically binds to Thymosin β16.

Polyclonal antibodies generated by the above technique may be used directly, or suitable antibody producing cells may be isolated from the animal and used to form a hybridoma by known means (Kohler and Milstein, Nature 256:795. (1975)). Selection of an appropriate hybridoma will also be apparent to those skilled in the art, and the resulting antibody may be used in a suitable assay to identify thymosin β16.

The term “antibody” as used herein encompasses polyclonal or monoclonal antibodies as well as functional fragments of antibodies, including fragments of chimeric, human, humanized, primatized, veneered or single-chain antibodies. Functional fragments include antigen-binding fragments which bind to thymosin β16. For example, antibody fragments capable of binding to thymosin β16 or portions thereof, including, but not limited to Fv, Fab, Fab′ and F (ab′) 2 fragments can be used. Such fragments can be produced by enzymatic cleavage or by recombinant techniques. For example, papain or pepsin cleavage can generate Fab or F (ab′) 2 fragments, respectively. Other proteases with the requisite substrate specificity can also be used to generate Fab or F (ab′) 2 fragments. Antibodies can also be produced in a variety of truncated forms using antibody genes in which one or more stop codons have been introduced upstream of the natural stop site. For example, a chimeric gene encoding a F (ab′) 2 heavy chain portion can be designed to include DNA sequences encoding the CH, domain and hinge region of the heavy chain.

Single-chain antibodies, and chimeric, human, humanized or primatized (CDR-grafted), or veneered antibodies, as well as chimeric, CDR-grafted or veneered single-chain antibodies, comprising portions derived from different species, and the like are also encompassed by the present invention and the term “antibody”. The various portions of these antibodies can be joined together chemically by conventional techniques, or can be prepared as a contiguous protein using genetic engineering techniques. For example, nucleic acids encoding a chimeric or humanized chain can be expressed to produce a contiguous protein. See, e.g., Cabilly et al., U.S. Pat. No. 4,816,567 Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Queen et al., European Patent No. 0451216 B1; and Padlan, E. A. et al., EP 0519596 A1. See also, Newman, R. et al., BioTechnology, 10: 1455-1460 (1992), regarding primatized antibody, and Ladner et al., U.S. Pat. No. 4,946,778 and Bird, R. E. et al., Science, 242: 423-426 (1988)) regarding single-chain antibodies.

Preparation of immunizing antigen, and polyclonal and monoclonal antibody production can be performed using any suitable technique. For example, monoclonal antibodies directed against binding cell surface epitopes can be readily produced by one skilled in the art. The general methodology for making monoclonal antibodies by hybridomas is well known. Other suitable methods of producing or isolating antibodies of the requisite specificity can be used, including, for example, methods which select recombinant antibody from a library (e.g., a phage display library).

In some embodiements, agents that specifically bind to Thymosin β16 other than antibodies are used, such as peptides. Peptides that specifically bind to Thymosin β16 can be identified by any means known in the art. For example, specific pepride binders of Thymosin β16 can be screened for using peptide phage display libraries.

Thymosin β16 Protein Detection Techniques

It is generally preferred to use antibodies, or antibody equivalents, to detect thymosin β16 protein. Methods for the detection of protein are well known to those skilled in the art, and include ELISA (enzyme linked immunosorbent assay), RIA (radioimmunoassay), Western blotting, and immunohistochemistry. Immunoassays such as ELISA or RIA, which can be extremely rapid, are more generally preferred. Antibody arrays or protein chips can also be employed, see for example U.S. Patent Application Nos: 20030013208A1; 20020155493A1, 20030017515 and U.S. Pat. Nos: 6,329,209; 6,365,418, herein incorporated by reference in their entirety.

Samples for diagnostic purposes may be obtained from any number of sources. A sample obtained directly from the tumor, such as the stroma or cytosol, may be used to determine the metastatic potential of the tumor. It may also be appropriate to obtain the sample from other biological specimens, such as blood, lymph nodes, or urine. Such diagnosis may be of particular importance in monitoring progress of a patient, such as after surgery to remove a tumor. If a reference reading is taken after the operation, then another taken at regular intervals, any rise could be indicative of a relapse, or possibly a metastasis.

ELISA and RIA procedures may be conducted such that a thymosin β16 standard is labeled (with a radioisotope such as ¹²⁵I or ³⁵S, or an assayable enzyme, such as horseradish peroxidase or alkaline phosphatase), and, together with the unlabelled sample, brought into contact with the corresponding antibody, whereon a second antibody is used to bind the first, and radioactivity or the immobilized enzyme assayed (competitive assay). Alternatively, thymosin β16 in the sample is allowed to react with the corresponding immobilized antibody, radioisotope- or enzyme-labeled anti-thymosin β16 antibody is allowed to react with the system, and radioactivity or the enzyme assayed (ELISA-sandwich assay). Other conventional methods may also be employed as suitable.

The above techniques may be conducted essentially as a “one-step” or “two-step” assay. The “one-step” assay involves contacting antigen with immobilized antibody and, without washing, contacting the mixture with labeled antibody. The “two-step” assay involves washing before contacting, the mixture with labeled antibody. Other conventional methods may also be employed as suitable.

Enzymatic and radiolabeling of thymosin β16 and/or the antibodies may be effected by conventional means. Such means will generally include covalent linking of the enzyme to the antigen or the antibody in question, such as by glutaraldehyde, specifically so as not to adversely affect the activity of the enzyme, by which is meant that the enzyme must still be capable of interacting with its substrate, although it is not necessary for all of the enzyme to be active, provided that enough remains active to permit the assay to be effected. Indeed, some techniques for binding enzyme are non-specific (such as using formaldehyde), and will only yield a proportion of active enzyme.

It is usually desirable to immobilize one component of the assay system on a support, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models and systems for which are well-known in the art. Simple polyethylene may provide a suitable support.

Enzymes employable for labeling are not particularly limited, but may be selected from the members of the oxidase group, for example. These catalyze production of hydrogen peroxide by reaction with their substrates, and glucose oxidase is often used for its good stability, ease of availability and cheapness, as well as the ready availability of its substrate (glucose). Activity of the oxidase may be assayed by measuring the concentration of hydrogen peroxide formed after reaction of the enzyme-labeled antibody with the substrate under controlled conditions well-known in the art.

Other techniques may be used to detect thymosin β16 according to a practitioner's preference based upon the present disclosure. One such technique is Western blotting (Towbin et at., Proc. Nat. Acad. Sci. 76:4350 (1979)), wherein a suitably treated sample is run on an SDS-PAGE gel before being transferred to a solid support, such as a nitrocellulose filter. Anti-thymosin β16 antibodies (unlabeled) are then brought into contact with the support and assayed by a secondary immunological reagent, such as labeled protein A or anti-immunoglobulin (suitable labels including ¹²⁵I, horseradish peroxidase and alkaline phosphatase). Chromatographic detection may also be used.

Immunohistochemistry may be used to detect expression of human thymosin β16 in a biopsy sample. A suitable antibody is brought into contact with, for example, a thin layer of cells, washed, and then contacted with a second, labeled antibody. Labeling may be by fluorescent markers, enzymes, such as peroxidase, avidin, or radiolabelling. The assay is scored visually, using microscopy.

In addition, the Thymosin β16 protein may be detected using Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See for example, U.S. Patent Application Nos: 20030199001, 20030134304, 20030077616, which are herein incorporated by reference.

Mass spectrometry methods are well known in the art and have been used to quantify and/or identify biomolecules, such as proteins (see, e.g., Li et al. (2000) Tibtech 18:151-160; Rowley et al. (2000) Methods 20: 383-397; and Kuster and Mann (1998) Curr. Opin. Structural Biol. 8: 393-400). Further, mass spectrometric techniques have been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000).

In certain embodiments, a gas phase ion spectrophotometer is used. In other embodiments, laser-desorption/ionization mass spectrometry is used to analyze the sample. Modem laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. However, MALDI has limitations as an analytical tool. It does not provide means for fractionating the sample, and the matrix material can interfere with detection, especially for low molecular weight analytes. See, e.g., U.S. Pat. No. 5,118,937 (Hillenkamp et al.), and U.S. Pat. No. 5,045,694 (Beavis & Chait).

In SELDI, the substrate surface is modified so that it is an active participant in the desorption process. In one variant, the surface is derivatized with adsorbent and/or capture reagents that selectively bind the protein of interest. In another variant, the surface is derivatized with energy absorbing molecules that are not desorbed when struck with the laser. In another variant, the surface is derivatized with molecules that bind the protein of interest and that contain a photolytic bond that is broken upon application of the laser. In each of these methods, the derivatizing agent generally is localized to a specific location on the substrate surface where the sample is applied. See, e.g., U.S. Pat. No. 5,719,060 (Hutchens & Yip) and WO 98/59361 (Hutchens & Yip). The two methods can be combined by, for example, using a SELDI affinity surface to capture an analyte and adding matrix-containing liquid to the captured analyte to provide the energy absorbing material.

For additional information regarding mass spectrometers, see, e.g., Principles of Instrumental Analysis, 3rd edition., Skoog, Saunders College Publishing, Philadelphia, 1985; and Kirk-Othmer Encyclopedia of Chemical Technology, 4.sup.th ed. Vol. 15 (John Wiley & Sons, New York 1995), pp. 1071-1094.

Detection of the presence of a marker or other substances will typically involve detection of signal intensity. This, in turn, can reflect the quantity and character of a polypeptide bound to the substrate. For example, in certain embodiments, the signal strength of peak values from spectra of a first sample and a second sample can be compared (e.g., visually, by computer analysis etc.), to determine the relative amounts of particular biomolecules. Software programs such as the Biomarker Wizard program (Ciphergen Biosystems, Inc., Fremont, Calif.) can be used to aid in analyzing mass spectra. The mass spectrometers and their techniques are well known to those of skill in the art.

Any person skilled in the art understands, any of the components of a mass spectrometer (e.g., desorption source, mass analyzer, detect, etc.) and varied sample preparations can be combined with other suitable components or preparations described herein, or to those known in the art. For example, in some embodiments a control sample may contain heavy atoms (e.g. ¹³C) thereby permitting the test sample to mixed with the known control sample in the same mass spectrometry run.

In one preferred embodiment, a laser desorption time-of-flight (TOF) mass spectrometer is used. In laser desorption mass spectrometry, a substrate with a bound marker is introduced into an inlet system. The marker is desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of molecules of specific mass to charge ratio.

In some embodiments the relative amounts of one or more biomolecules present in a first or second sample is determined, in part, by executing an algorithm with a programmable digital computer. The algorithm identifies at least one peak value in the first mass spectrum and the second mass spectrum. The algorithm then compares the signal strength of the peak value of the first mass spectrum to the signal strength of the peak value of the second mass spectrum of the mass spectrum. The relative signal strengths are an indication of the amount of the biomolecule that is present in the first and second samples. A standard containing a known amount of a biomolecule can be analyzed as the second sample to provide better quantify the amount of the biomolecule present in the first sample. In certain embodiments, the identity of the biomolecules in the first and second sample can also be determined.

Thymosin β16 Detection Kit

This invention provides a convenient kit for measuring human thymosin β16. This kit includes antibodies or antibody fragments which selectively bind human thymosin β16 or a set of DNA oligonucleotide primers that allows synthesis of cDNA encoding the protein or a DNA probe that detects expression of thymosin β16 mRNA. Preferably, the primers and probes comprise at least 17 nucleotides and hybridize under stringent conditions to a DNA fragment having the nucleotide sequence set forth in SEQ ID NOS: 2 or 3. As herein used, the term “stringent conditions” means hybridization will occur only if there is at least 95% and preferably at least 97% identity between the sequences.

Thymosin β16 DNA and Protein Production

DNA encoding thymosin β16 and recombinant thymosin β16 may be produced according to the methods known in the art.

The thymosin β16 protein may be isolated from a variety of sources, such as from human tissue types or from another source, or prepared by recombinant or synthetic methods. Proteins can be produced by well known synthetic methods including Merrifield solid-phase synthesis, t-Boc-based synthesis, Fmoc synthesis and variations on these techniques (See, for example, Atherton and Sheppard, Solid Phase Peptide Synthesis: A Practical Approach, New York: IRL Press, 1989; Stewart and Young: Solid-Phase Peptide Synthesis 2nd Ed., Rockford, Ill.: Pierce Chemical Co., 1984; and Jones, The Chemical Synthesis of Peptides, Oxford: Clarendon Press, 1994). Recombinant methods are one preferred method to produce the thymosin β16 protein. A wide variety of molecular and biochemical methods are available for generating and expressing the thymosin β16; see e.g. the procedures disclosed in Molecular Cloning, A Laboratory Manual (2nd Ed., Sambrook, Fritsch and Maniatis, Cold Spring Harbor), Current Protocols in Molecular Biology (Eds. Ausubel, Brent, Kingston, More, Feidman, Smith and Stuhl, Greene Publ. Assoc., Wiley-Interscience, NY, N.Y. 1992) or other procedures that are otherwise known in the art.

Thymosin β16 Cloning

Where it is desired to express the protein or a fragment thereof, any suitable system can be used. The general nature of suitable vectors, expression vectors and constructions therefor will be apparent to those skilled in the art.

Suitable expression vectors may be based on phages or plasmids, both of which are generally host-specific, although these can often be engineered for other hosts. Other suitable vectors include cosmids and retroviruses, and any other vehicles, which may or may not be specific for a given system. Control sequences, such as recognition, promoter, operator, inducer, terminator and other sequences essential and/or useful in the regulation of expression, will be readily apparent to those skilled in the art.

Correct preparation of nucleotide sequences may be confirmed, for example, by the method of Sanger et al. (Proc. Natl. Acad. Sci. USA 74:5463-7 (1977)).

A DNA fragment encoding the thymosin β16 or a fragment thereof, may readily be inserted into a suitable vector. Ideally, the receiving vector has suitable restriction sites for ease of insertion, but blunt-end ligation, for example, may also be used, although this may lead to uncertainty over reading frame and direction of insertion. In such an instance, it is a matter of course to test transformants for expression, 1 in 6 of which should have the correct reading frame. Suitable vectors may be selected as a matter of course by those skilled in the art according to the expression system desired.

Thymosin β16 Protein Production

Isolated thymosin β16 protein and fragments thereof may be produced using any expression system known to those skilled in the art. Such suitable expression systems include bacteria, such as E. coli, and eukaryotes, such as yeast, baculovirus, insect or mammalian cell-based expression systems, etc., depending on the size, nature and quantity of the polypeptide.

The term “isolated” means that the polypeptide is removed from its original environment. For example, a naturally-occurring polynucleotide or polypeptide present in a living animal is not isolated, but the same polynucleotide or DNA or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment.

By transforming a suitable bacterial or eukaryotic organism, and preferably, a eukaryotic cell line, such as HeLa cells, with the plasmid obtained, selecting the eukaryotic transformant with geneticin, zeocin, blasticin or a similar compound (or ampicillin in the case of a bacterial transformant) or by other suitable means if required, and adding tryptophan or other suitable promoter-inducer if necessary, the desired polypeptide or protein may be expressed. The extent of expression may be analyzed by SDS polyacrylamide gel electrophoresis-SDS-PAGE (Laemelli, Nature 227:680-685 (1970)).

Suitable methods for growing and transforming cultures are usefully illustrated in, for example, Maniatis (Molecular Cloning, A Laboratory Notebook, Maniatis et al. (eds.), Cold Spring Harbor Labs, N.Y. (1989)).

Cultures useful for production of polypeptides or proteins may suitably be cultures of any living cells, and may vary from prokaryotic expression systems up to eukaryotic expression systems. One preferred prokaryotic system is that of E. coli, owing to its ease of manipulation. However, it is also possible to use a higher system, such as a mammalian cell line, for expression of a eukaryotic protein. Currently preferred cell lines for transient expression are the HeLa and Cos cell lines. Other expression systems include the Chinese Hamster Ovary (CHO) cell line and the baculovirus system.

Other expression systems which may be employed include streptomycetes, for example, and yeasts, such as Saccharomyces spp., especially S. cerevisiae. Any system may be used as desired, generally depending on what is required by the operator. Suitable systems may also be used to amplify the genetic material, but it is generally convenient to use E. coli for this purpose when only proliferation of the DNA is required.

The polypeptides and proteins may be isolated from the fermentation or cell culture and purified using any of a variety of conventional methods including: liquid chromatography such as normal or reversed phase, using HPLC, FPLC and the like; affinity chromatography (such as with inorganic ligands or monoclonal antibodies); size exclusion chromatography; immobilized metal chelate chromatography; gel electrophoresis; and the like. One of skill in the art may select the most appropriate isolation and purification techniques without departing from the scope of this invention.

Therapeutic Applications Using Thymosin β16

The presence of thymosin β16 protein is positively correlated with metastasis. Therefore, thymosin β16 protein could be useful in therapeutic and diagnostic applications. For example, therapeutic approaches include the use of antibodies to block thymosin β16 protein, the use of antibodies for imaging applications, antisense, and RNAi technology to block thymosin β16 expression, membrane localization for tumor targeting and delivery of therapeutics to tumor cells, and immunotherapies such as vaccines. Diagnostically, the cleavage of ectodomain of the thymosin β16 protein, easily detected in the blood serum or urine, can be used as a marker for metastic cancer.

Thymosin β16 Blocking Antibodies or Aptamers

One can treat a range of afflictions or diseases associated with expression of the protein by directly blocking the protein. This can be accomplished by a range of different approaches, including the use of antibodies, small molecules, and antagonists. One preferred approach is the use of antibodies that specifically block activity of the protein. Aptemers may also be used.

In accordance with yet another aspect of the present invention, there are provided isolated antibodies or antibody fragments which selectively bind the protein. The antibody fragments include, for example, Fab, Fab′, F(ab′)2 or Fv fragments. The antibody may be a single chain antibody, a humanized antibody or a chimeric antibody.

Antibodies, or their equivalents, or other thymosin β16 antagonists may also be used in accordance with the present invention for the treatment or prophylaxis of cancers. Administration of a suitable dose of the antibody or the antagonist may serve to block the activity of the protein and this may provide a crucial time window in which to treat the malignant growth.

Prophylaxis may be appropriate even at very early stages of the disease, as it is not known what specific event actually triggers metastasis in any given case. Thus, administration of the antibodies, their equivalents, intrabodies, antagonists which interfere with protein activity, may be effected as soon as cancer is diagnosed, and treatment continued for as long as is necessary, preferably until the threat of the disease has been removed. Such treatment may also be used prophylactically in individuals at high risk for development of certain cancers, e.g., prostate or breast.

A method of treatment involves attachment of a suitable toxin to the antibodies which then target the area of the tumor. Such toxins are well known in the art, and may comprise toxic radioisotopes, heavy metals, enzymes and complement activators, as well as such natural toxins as ricin which are capable of acting at the level of only one or two molecules per cell. It may also be possible to use such a technique to deliver localized doses of suitable physiologically active compounds, which may be used, for example, to treat cancers.

It will be appreciated that antibodies for use in accordance with the present invention, whether for diagnostic or therapeutic applications, may be monoclonal or polyclonal as appropriate. Antibody equivalents of these may comprise: the Fab′ fragments of the antibodies, such as Fab, Fab′, F(ab′)2 and Fv; idiotopes; or the results of allotope grafting (where the recognition region of an animal antibody is grafted into the appropriate region of a human antibody to avoid an immune response in the patient), for example. Single chain antibodies may also be used. Other suitable modifications and/or agents will be apparent to those skilled in the art.

Chimeric and humanized antibodies are also within the scope of the invention. It is expected that chimeric and humanized antibodies would be less immunogenic in a human subject than the corresponding non-chimeric antibody. A variety of approaches for making chimeric antibodies, comprising for example a non-human variable region and a human constant region, have been described. See, for example, Morrison et al., Proc. Natl. Acad. Sci. U.S.A. 81,6851 (1985); Takeda, et al., Nature 314,452(1985), Cabilly et al., U.S. Pat. No. 4,816,567; Boss et al., U.S. Pat. No. 4,816,397; Tanaguchi et al., European Patent Publication EP 171496; European Patent Publication 0173494, United Kingdom Patent GB 2177096B. Additionally, a chimeric antibody can be further “humanized” such that parts of the variable regions, especially the conserved framework regions of the antigen-binding domain, are of human origin and only the hypervariable regions are of non-human origin. Such altered immunoglobulin molecules may be made by any of several techniques known in the art, (e.g., Teng et al., Proc. Natl. Acad Sci. U.S.A., 80, 7308-7312 (1983); Kozbor et al., Immunology Today, 4, 7279 (1983); Olsson et al., Meth. Enzymol., 92, 3-16 (1982)), and are preferably made according to the teachings of PCT Publication WO92/06193 or EP 0239400. Humanized antibodies can be commercially produced by, for example, Scotgen Limited, 2 Holly Road, Twickenham, Middlesex, Great Britain.

In addition to using antibodies to inhibit thymosin β16, it may also be possible to use other forms of inhibitors. For example, it may be possible to identify antgonists that functionally inhibit thymosin β16. In addition, it may also be possible to interfere with the binding of thymosin β16 to target proteins, Other suitable inhibitors will be apparent to the skilled person.

The present invention further provides use of the thymosin β16 for intracellular or extracellular targets to affect activity. Intracellular targeting can be accomplished through the use of intracellularly expressed antibodies referred to as intrabodies.

The antibody (or other inhibitors or intrabody) can be administered by a number of methods. One preferred method is set forth by Marasco and Haseltine in PCT WO94/02610, which is incorporated herein by reference. This method discloses the intracellular delivery of a gene encoding the antibody. One would preferably use a gene encoding a single chain antibody. The antibody would preferably contain a nuclear localization sequence. One preferably uses an SV40 nuclear localization signal. By this method one can intracellularly express an antibody, which can block thymosin β16 functioning in desired cells.

Where the present invention provides for the administration of, for example, antibodies to a patient, then this may be by any suitable route. If the tumor is still thought to be, or diagnosed as, localized, then an appropriate method of administration may be by injection direct to the site. Administration may also be by injection, including subcutaneous, intramuscular, intravenous and intradermal injections.

Aptamer can be produced using the methodology disclosed in a U.S. Pat. No. 5,270,163 and WO 91/19813.

Formulations may be any that are appropriate to the route of administration, and will be apparent to those skilled in the art. The formulations may contain a suitable carrier, such as saline, and may also comprise bulking agents, other medicinal preparations, adjuvants and any other suitable pharmaceutical ingredients. Catheters are one preferred mode of administration.

Imaging Techniques

Anti-thymosin β16 antibodies may also be used for imaging purposes, for example, to detect tumor metastasis. Suitable labels include radioisotopes, iodine (¹²⁵I, ¹²¹I), carbon (¹⁴C), sulphur (³⁵S), tritium (³H), indium (¹¹²In), and technetium (^{99m}Tc), fluorescent labels, such as fluorescein and rhodamine, and biotin.

However, for in vivo imaging purposes, the position becomes more restrictive, as antibodies are not detectable, as such, from outside the body, and so must be labeled, or otherwise modified, to permit detection. Markers for this purpose may be any that do not substantially interfere with the antibody binding, but which allow external detection. Suitable markers may include those that may be detected by X-radiography, NMR or MRI. For X-radiographic techniques, suitable markers include any radioisotope that emits detectable radiation but that is not overtly harmful to the patient, such as barium or caesium, for example. Suitable markers for NMR and MRI generally include those with a detectable characteristic spin, such as deuterium, which may be incorporated into the antibody by suitable labeling of nutrients for the relevant hybridoma, for example.

The size of the subject, and the imaging system used, will determine the quantity of imaging moiety needed to produce diagnostic images. In the case of a radioisotope moiety, for a human subject, the quantity of radioactivity injected will normally range from about 5 to 20 millicuries of technetium-99 m. The labeled antibody or antibody fragment will then preferentially accumulate at the location of cells which contain thymosin β16. The labeled antibody or antibody fragment can then be detected using known techniques.

Antisense Technology

Thymosin β16 expression may also be inhibited in vivo by the use of antisense technology. Gene expression can be controlled through triple-helix formation or antisense DNA or RNA, both of which methods are based on binding of a polynucleotide to DNA or RNA. An antisense nucleic acid molecule which is complementary to a nucleic acid molecule encoding thymosin β16 can be designed based upon the isolated nucleic acid molecules encoding thymosin β16. An antisense nucleic acid molecule can comprise a nucleotide sequence which is complementary to a coding strand of a nucleic acid, e.g. complementary to an mRNA sequence, constructed according to the rules of Watson and Crick base pairing, and can hydrogen bond to the coding strand of the nucleic acid. The antisense sequence complementary to a sequence of an mRNA can be complementary to a sequence in the coding region of the mRNA or can be complementary to a 5′ or 3′ untranslated region of the mRNA. Furthermore, an antisense nucleic acid can be complementary in sequence to a regulatory region of the gene encoding the mRNA, for instance a transcription initiation sequence or regulatory element. Preferably, an antisense nucleic acid complementary to a region preceding or spanning the initiation codon or in the 3′ untranslated region of an mRNA is used. An antisense nucleic acid can be designed based upon the nucleotide sequence shown in SEQ ID NO:2 or 3 (thymosin β16). A nucleic acid is designed which has a sequence complementary to a sequence of the coding or untranslated region of the shown nucleic acid. Alternatively, an antisense nucleic acid can be designed based upon sequences of the thymosin β16 gene, which can be identified by screening a genomic DNA library with an isolated nucleic acid of the invention. For example, the sequence of an important regulatory element can be determined by standard techniques and a sequence which is antisense to the regulatory element can be designed.

The antisense nucleic acids and oligonucleotides of the invention can be constructed using chemical synthesis and enzymatic ligation reactions using procedures known in the art. The antisense nucleic acid or oligonucleotide can be chemically synthesized using naturally occurring nucleotides or variously modified nucleotides designed to increase the biological stability of the molecules or to increase the physical stability of the duplex formed between the antisense and sense nucleic acids e.g. phosphorothioate derivatives and acridine substituted nucleotides can be used. Alternatively, the antisense nucleic acids and oligonucleotides can be produced biologically using an expression vector into which a nucleic acid has been subcloned in an antisense orientation (i.e. nucleic acid transcribed from the inserted nucleic acid will be of an antisense orientation to a target nucleic acid of interest). The antisense expression vector is introduced into cells in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub, H. et al., Antisense RNA as a molecular tool for genetic analysis, Reviews—Trends in Genetics, Vol. 1 (1)1986.

In addition, ribozymes can be used to inhibit in vitro expression of thymosin β16. For example, the nucleic acids of the invention can further be used to design ribozymes which are capable of cleaving a single-stranded nucleic acid encoding a thymosin β16 protein, such as a thymosin β16 mRNA transcript. A catalytic RNA (ribozyme) having ribonuclease activity can be designed which has specificity for an mRNA encoding thymosin β16 based upon the sequence of a nucleic acid of the invention (e.g., SEQ ID NO: 1). For example, a derivative of a Tetrahymena L-19 IVS RNA can be constructed in which the base sequence of the active site is complementary to the base sequence to be cleaved in a thymosin β16-encoding mRNA. See for example Cech, et al., U.S. Pat. No. 4,987,071; Cech, et al., U.S. Pat. No. 5,116,742. Alternatively, a nucleic acid of the invention could be used to select a catalytic RNA having a specific ribonuclease activity from a pool of RNA molecules. See for example Bartel, D. and Szostak, J. W. Science 261: 1411-1418 (1993). RNA-mediated interference (RNAi) (Fire, et al., Nature 391: 806-811, 1998) may also be used.

RNAi Technology

RNA interference or “RNAi” is a term initially coined by Fire and co-workers to describe the observation that double-stranded RNA (dsRNA) can block gene expression when it is introduced into worms (Fire et al. (1998) Nature 391, 806-811). dsRNA directs gene-specific, post-transcriptional silencing in many organisms, including vertebrates, and has provided a new tool for studying gene function. RNAi involves mRNA degradation of a target gene. Results showed that RNAi is ATP-dependent yet uncoupled from mRNA translation. That is, protein synthesis is not required for RNAi in vitro. In the RNAi reaction, both strands (sense and antisense) of the dsRNA are processed to small RNA fragments or segments of from about 21 to about 23 nucleotides (nt) in length (RNAs with mobility in sequencing gels that correspond to markers that are 21-23 nt in length, optionally referred to as 21-23 nt RNA). Processing of the dsRNA to the small RNA fragments does not require the targeted mRNA, which demonstrates that the small RNA species is generated by processing of the dsRNA and not as a product of dsRNA-targeted mRNA degradation. The mRNA is cleaved only within the region of identity with the dsRNA. Cleavage occurs at sites 21-23 nucleotides apart, the same interval observed for the dsRNA itself, suggesting that the 21-23 nucleotide fragments from the dsRNA are guiding mRNA cleavage. Isolated RNA molecules (double-stranded; single-stranded) of from about 21 to about 23 nucleotides mediate RNAi. That is, the isolated RNAs mediate degradation of mRNA of a gene to which the mRNA corresponds (mediate degradation of mRNA that is the transcriptional product of the gene, which is also referred to as a target gene). Isolated RNA molecules specific to thymosin β16 mRNA, which mediate RNAi, are antagonists useful in the method of the present invention. See for example U.S. Patent Application Nos: 20030153519A1; 20030167490A1; and U.S. Pat. Nos: 6,506,559; 6,573,099, which are herein incorporated by reference in their entirety.

The term “pharmaceutically acceptable” refers to compounds and compositions which may be administered to mammals without undue toxicity. Exemplary pharmaceutically acceptable salts include mineral acid salts such as hydrochlorides, hydrobromides, phosphates, sulfates, and the like; and the salts of organic acids such as acetates, propionates, malonates, benzoates, and the like.

The antibodies, nucleic acids or antagonists of the invention are administered orally, topically, or by parenteral means, including subcutaneous and intramuscular injection, implantation of sustained release depots, intravenous injection, intranasal administration, and the like. Accordingly, antibodies or nucleic acids of the invention may be administered as a pharmaceutical composition comprising the antibody or nucleic acid of the invention in combination with a pharmaceutically acceptable carrier. Such compositions may be aqueous solutions, emulsions, creams, ointments, suspensions, gels, liposomal suspensions, and the like. Suitable carriers (excipients) include water, saline, Ringer's solution, dextrose solution, and solutions of ethanol, glucose, sucrose, dextran, mannose, mannitol, sorbitol, polyethylene glycol (PEG), phosphate, acetate, gelatin, collagen, Carbopol Registered TM, vegetable oils, and the like. One may additionally include suitable preservatives, stabilizers, antioxidants, antimicrobials, and buffering agents, for example, BHA, BHT, citric acid, ascorbic acid, tetracycline, and the like. Cream or ointment bases useful in formulation include lanolin, Silvadene Registered TM (Marion), Aquaphor Registered TM (Duke Laboratories), and the like. Other topical formulations include aerosols, bandages, and other wound dressings. Alternatively one may incorporate or encapsulate the compounds in a suitable polymer matrix or membrane, thus providing a sustained-release delivery device suitable for implantation near the site to be treated locally. Other devices include indwelling catheters and devices such as the Alzet Registered TM minipump. Ophthalmic preparations may be formulated using commercially available vehicles such as Sorbi-care Registered TM (Allergan), Neodecadron Registered TM (Merck, Sharp & Dohme), Lacrilube Registered TM, and the like, or may employ topical preparations such as that described in U.S. Pat. No. 5,124,155, incorporated herein by reference. Further, one may provide an antagonist in solid form, especially as a lyophilized powder. Lyophilized formulations typically contain stabilizing and bulking agents, for example human serum albumin, sucrose, mannitol, and the like. A thorough discussion of pharmaceutically acceptable excipients is available in Remington's Pharmaceutical Sciences (Mack Pub. Co.).

The amount of antibody, nucleic acid or inhibitor required to treat any particular disorder will of course vary depending upon the nature and severity of the disorder, the age and condition of the subject, and other factors readily determined by one of ordinary skill in the art.

Immunotherapy

In further aspects, the present invention provides methods for using thymosin β16 or an immunoreactive polypeptide thereof (or DNA encoding the protein or polypeptides) for immunotherapy of cancer in a patient, preferably prostate cancer. As used herein, a “patient” refers to any warm-blooded animal, preferably a human. A patient may be afflicted with a disease, or may be free of detectable disease. Accordingly, thymosin β16 or an immunoreactive polypeptide thereof may be used to treat cancer or to inhibit the development of cancer.

In accordance with this method, the protein, polypeptide or DNA is generally present within a pharmaceutical composition and/or a vaccine. Pharmaceutical compositions may comprise the full length protein or one or more immunogenic polypeptides, and a physiologically acceptable carrier. The vaccines may comprise the full length protein or one or more immunogenic polypeptides and a non-specific immune response enhancer, such as an adjuvant, biodegradable microsphere (PLG) or a liposome (into which the polypeptide is incorporated).

Alternatively, a pharmaceutical composition or vaccine may contain DNA encoding thymosin β16 or an immunogenic polypeptide thereof, such that the full length protein or polypeptide is generated in situ. In such pharmaceutical compositions and vaccines, the DNA may be present within any of a variety of delivery systems known to those of ordinary skill in the art, including nucleic acid expression systems, bacteria and viral expression systems. Appropriate nucleic acid expression systems contain the necessary DNA sequences for expression in the patient (such as a suitable promoter). Bacterial delivery systems involve the administration of a bacterium (such as Bacillus-Calmette-Guerrin) that expresses an epitope of a prostate cell antigen on its cell surface. In a preferred embodiment, the DNA may be introduced using a viral expression system (e.g., vaccinia or other pox virus, retrovirus, or adenovirus), which may involve the use of a non-pathogenic (defective), replication competent virus. Suitable systems are disclosed, for example, in Fisher-Hoch et al., PNAS 86:317-321, 1989; Flexner et al., Ann. N.Y Acad. Sci. 569:86-103, 1989; Flexner et al., Vaccine 8:17-21, 1990; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, iotechniques 6:616-627,1988; Rosenfeld et al., Science 252:431-434, 1991; Kolls et al., PNAS 91:215-219, 1994; Kass-Eisler et al., PNAS 90:11498-11502, 1993; Guzman et al., Circulation 88:2838-2848, 1993; and Guzman et al., Cir. Res. 73:1202-1207, 1993. Techniques for incorporating DNA into such expression systems are well known to those of ordinary skill in the art. The DNA may also be “naked,” as described, for example, in published PCT application WO 90/11092, and Ulmer et al., Science 259:1745-1749 (1993), reviewed by Cohen, Science 259:1691-1692 (1993).

Routes and frequency of administration, as well as dosage, will vary from individual to individual and may parallel those currently being used in immunotherapy of other diseases. In general, the pharmaceutical compositions and vaccines may be administered by injection (e.g., intracutaneous, intramuscular, intravenous or subcutaneous), intranasally (e.g., by aspiration) or orally. Between 1 and 10 doses may be administered over a 3-24 week period. Preferably, 4 doses are administered, at an interval of 3 months, and booster administrations may be given periodically thereafter. Alternate protocols may be appropriate for individual patients. A suitable dose is an amount of polypeptide or DNA that is effective to raise an immune response (cellular and/or humoral) against tumor cells, e.g., prostate tumor cells, in a treated patient. A suitable immune response is at least 10-50% above the basal (i.e. untreated) level. In general, the amount of polypeptide present in a dose (or produced in situ by the DNA in a dose) ranges from about 1 pg to about 100 mg per kg of host, typically from about 10 pg to about 1 mg, and preferably from about 100 pg to about 1 μg. Suitable dose sizes will vary with the size of the patient, but will typically range from about 0.01 mL to about 5 ml.

Thymosin β16 or an immunogenic polypeptide thereof can be used in cell based immunotherapies, i.e. stimulation of dendritic cells with thymosin β16 or fusion with thymosin β16 expressing cells. The modified dendritic cells, once injected into the patient, are a cellular vaccine, where the dendritic cells activate an immune response against the thymosin β16 expressing cancer.

While any suitable carrier known to those of ordinary skill in the art may be employed in the pharmaceutical compositions of this invention, the type of carrier will vary depending on the mode of administration. For parenteral administration, such as subcutaneous injection, the carrier preferably comprises water, saline, alcohol, a fat, a wax and/or a buffer. For oral administration, any of the above carriers or a solid carrier, such as mannitol, lactose, starch, magnesium stearate, sodium saccharine, talcum, cellulose, glucose, sucrose, and/or magnesium carbonate, may be employed. Biodegradable microspheres (e.g., polyleptic galactide) may also be employed as carriers for the pharmaceutical compositions of this invention. Suitable biodegradable microspheres are disclosed, for example, in U.S. Pat. Nos. 4,897,268 and 5,075,109.

Any of a variety of non-specific immune response enhancers may be employed in the vaccines of this invention. For example, an adjuvant may be included. Most adjuvants contain a substance designed to protect the antigen from rapid catabolism, such as aluminum hydroxide or mineral oil, and a nonspecific stimulator of immune response, such as lipid A, Bordella pertussis or Mycobacterium tuberculosis. Such adjuvants are commercially available as, for example. Freund's Incomplete Adjuvant and Complete Adjuvant (Difco Laboratories. Detroit, Mich.) and Merck Adjuvant 65 (Merck and Company, Inc., Rahway, N.J.).

All references cited above or below are herein incorporated by reference.

The present invention is further illustrated by the following Example. This Example is provided to aid in the understanding of the invention and are not construed as a limitation thereof.

### EXAMPLES

### Example 1

Following the discovery of Tβ15 searches NCBI expressed tag (EST) data base were performed to determine if other mammalian species possess a Tβ15 ortholog. These searches uncovered a human EST (Hillier et al. 1995, unpublished, genebank accession R08518) encoding a novel β-thymosin protein, with many of the same characteristics displayed by Tβ15. Using the EST data, we proceeded to clone the complete cDNA from human prostatic carcinoma LnCap cells, and have confirmed that the amino acid sequence deduced from the cDNA sequence is correct.

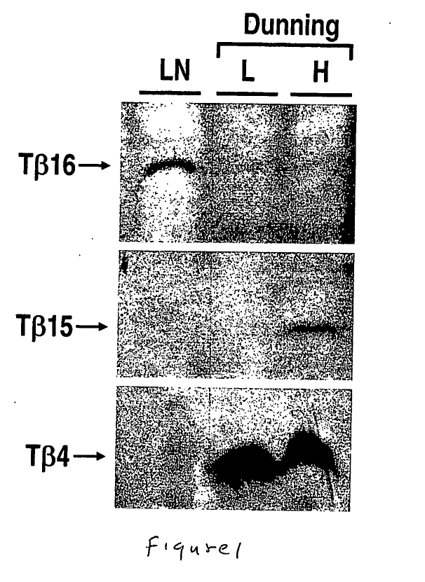

In FIG. 1 we examined the level of endogenous β-thymosin proteins (Tβ4, Tβ15, Tβ16) in the LnCaP human prostate cancer cell line, originally derived from a lymph node metastasis, and in low or high metastatic variants of the Dunning rat prostatic carcinoma cell line. Western blost analysis revealed that LnCaP cells produce thymosin Tβ16 but not thymosin Tβ4 or Tβ15. In contrast the high metastatic variant of the Dunning rat prostatic carcinoma contains Tβ4 and Tβ15 but not Tβ16, whereas the Tβ4 is the only form of β thymosin found in the nonmetastatic variant. This is the first time the endogenous Tβ16 protein has been detected in any cell type.

### Example 2

Immunohistochemistry—Summary of Tβ16 Expression in Human Prostatic Carcinoma.

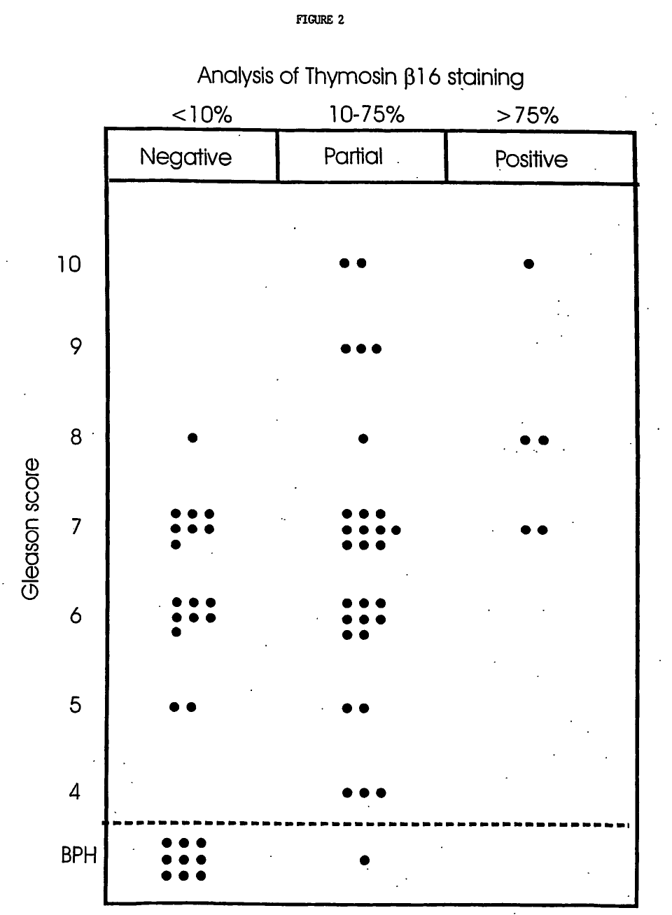

The results (FIG. 2) show a general correlation between thymosin β16 staining and the Gleason score. For instance, a higher percentage of high-grade tumors exhibited partial or positive staining (90%, Gleason score 8-10) than did medium-grade tumors (59%, Gleason score 6-7), or low grade tumors (Gleason score 2-5). It is important to note that medium-grade prostatic carcinomas (Gleason scores 6-7) are found in all three categories. Presently, Gleason scores of 6 to 7 have no predictive value regarding distant failure in patients with clinically localized prostate cancer. As such, thymosin β16 is an independent predictor of distant failure for patients with moderately differentiated prostate carcinomas. By contrast, Tβ16 staining was absent in nine specimens of benign prostatic hyperplasia (BPH). In the one case of BPH where a Tβ16 signal was observed, staining was weak and limited to a single prostate gland. Moreover, prostate specific antigen (PSA) is considered the gold standard of prostate cancer diagnosis, yet PSA is highly expressed in BPH tissues.

### Example 3

β-Thymosin RNA Expression Profile in Low and High Metastatic Cell Lines.

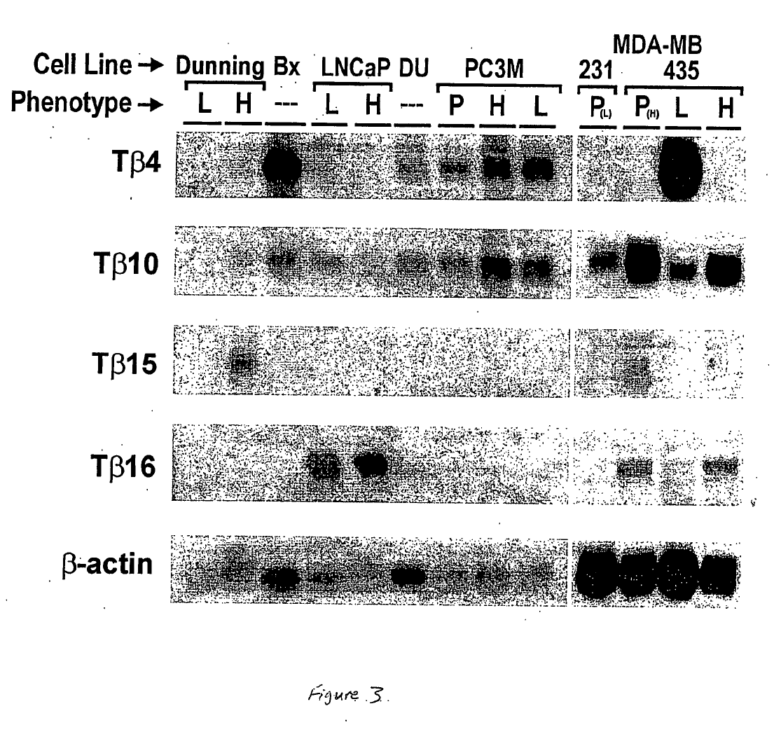

Northern blot analysis of β-thymosin RNA expression in low and high metastatic cell lines was performed. FIG. 3 demonstrates that a changes in metastatic phenotype alters the β-thymosin expression profile in tumor cells. β-thymosin isoforms Tβ15 and Tβ16 are found primarly in highly metastatic cells including those that originate from prostate or breast carcinomas. For instance Tβ16 levels are higher in subclones of LnCaP prostate carcinoma cells and MDA-MB435 breast carcinoma cells selected for increased metastatic phenotype. Like Tβ15 or Tβ16, thymosin β10 also rose with increasing metastatic phenotype in many cell types (e.g. Dunning, PC3M, MDA-MB), but decreased in others (e.g. LnCaP). By contrast, levels of the thymosin β4 isoform tend to be highest only in cells that produce fewer metastases (e.g. PC3M-L & MDA-MB-435-L) than parental cell lines or the highly metastatic variants.

In FIG. 3, DU stands for DU 145 which is a prostate cancer cell line from a lesion in the brain of a patient with metastatic carcinoma of the prostate. PC3M is a derviative of the PC3 cell line, which is a prostate cancer cell line isolated from a bone metastasis of a grade IV prostatic adenocarcinoma from a 62-year-old male Caucasian. PC3M was reisolated as a cell line from a tumor metastasis which formed in a SCID mouse. BxPC-3 (or Bx) is a cell line isolated from a pancreatic carcinoma.

### Example 4

Tβ16 is Expressed at Low Levels in Normal Human Adult Tissues and Expression Increases in a Variety of Tumors.

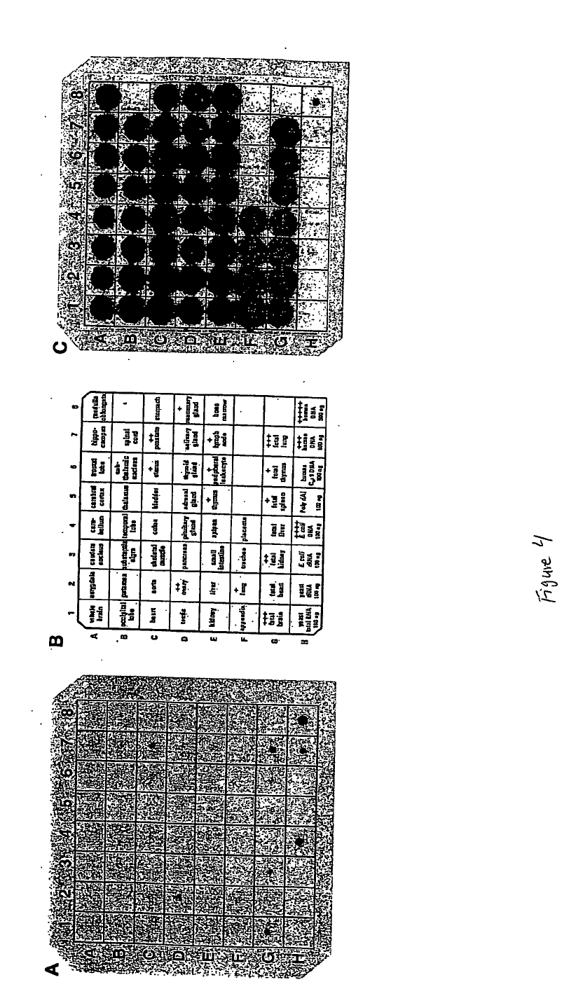

Tβ16 mRNA expression in normal human adult and fetal tissues was monitored (FIG. 4). The results show that Tβ16 mRNA is absent or expressed at low levels in normal adult tissues compared to the endogenous levels of β-actin mRNA which encodes the primary β-thymosin binding partner (FIG. 4A vs. FIG. 4C). Moderate levels of Tβ15 expression were observed in normal tissues from brain and fetal lung. Like β-actin, thymosin β16 belongs to a multigene family with multiple pseudogenes and the signal from the genomic DNA control spots reflects hybridization to the genomic alleles. However, these hybridization control spots in the Tβ16 blot yielded much stronger signals than any normal tissue, whereas tissues accounted for the vast majority of signal on the β-actin blot. Even in tissues with detectable levels of Tβ16 mRNA (e.g. prostate, ovary), when taken together this data indicates that Tβ16 is expressed at low levels in normal human adult tissues.

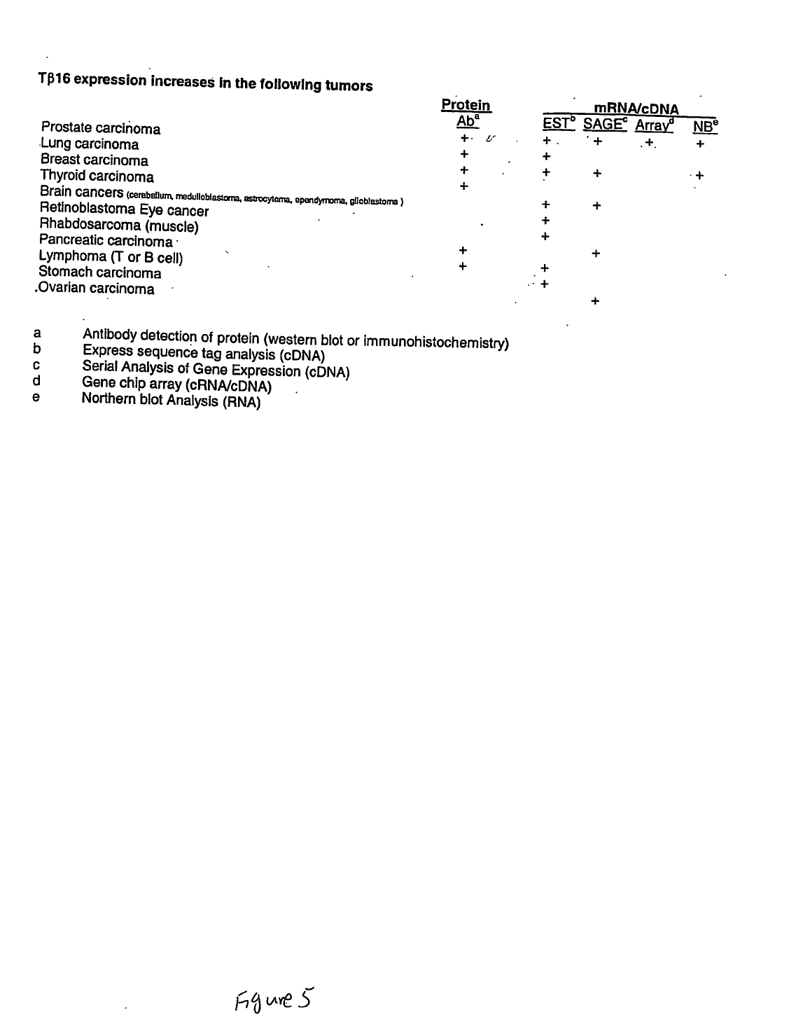

The levels of Tβ16 assessed in a variety of tumors. FIG. 5 shows that increased expression of Tβ16 was found in Prostate cancer, Lung carcinoma, Breast carcinoma, Thyroid carcinoma, Brain cancers (cerebellum, medulloblastoma, astrocytoma, ependymoma, glioblastoma), Eye cancer (retinoblastoma), Muscle (rhabdosarcoma), Pancreatic cancer, Lymphoma (T or B cell), Stomach cancer, and Ovarian carcinoma, as determined by either (a) Antibody staining (b) Express Sequence Tag analysis (c) Serial Analysis of Gene Expression (SAGE) (d) Gene Chip Array (e) or Northern Blot analysis.

### Example 5

Expression of β-Thymosin Isoforms Tβ4 and Tβ16 in Various Human Tissues by Northern Blot Analysis.

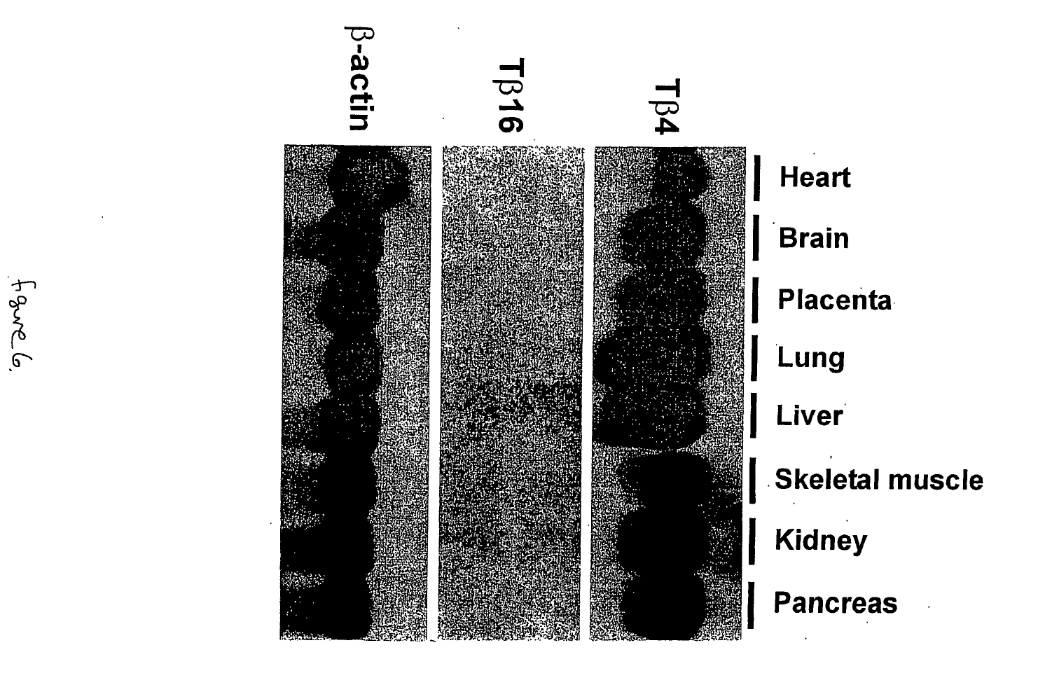

A multiple-tissue Northern blot of normal human tissue (Clontech, PT1200-1) was sequentially hybridized with Tβ16 and Tβ4 ³²P labeled DNA probes (FIG. 6). β-actin is shown as a loading control. Thymosin β16 mRNA could not be detected in any of the normal tissues present on the multilple-tissue northern blot (Clontech, Calif.). In contrast, Tβ04 a ubiquitous β-thymosin isoform, was highly expressed in all tissues, with the exception of muscle tissues which contained moderate amounts of the Tβ4 mRNA. Similar quantities of the β-actin loading control were observed in all tissues, although other highly conserved actin isoforms are probably responsible for the signal in muscle tissues.

The references cited throughout the specification are incorporated herein by reference. The present invention has been described with reference to specific embodiments. However, this application is intended to cover those changes and substitutions that may be made by those skilled in the art, without departing from the spirit and the scope of the appended claims.

TB16protein

(SEQ ID No:1)

MSDKPDLSEVEKFDRSKLKKTNTEEKNTLPSKETIQQEKECVQTS

TB16 RNA allele1

(SEQ ID NO:2)

GCGGGAAGGCTAACCTGGTGCGGAGCCAGCCTGGGTCTCAGCCCCGCGTA
CGGCCTTTCACGAGTCTTCAAGCCTTCAGGCTTTCTTCTAGTCAAGATGA
GTGATAAACCAGACTTGTCGGAAGTGGAGAAGTTTGACAGGTCAAAACTG
AAGAAAACTAATACTGAAGAAAAAAATACTCTTCCCTCAAAGGAAACTAT
CCAGCAGGAGAAAGAGTGTGTTCAAACATCATAAAATGGGGATCTCCTCC
AAAGAGCAGATTTCAGCATTGCCTGACAGTCTTGGTTTTAGGCTTGTTTT
TTTGTAAACCTGTGTGTTTGTAGAGATTTCAGACATCTTCTGATTTCTTC
TCACCTATATTCCCTGGTTAAGAGGTCAGGGGTAGTGAATGTTTCCTTAA
GTTCCTTTTTAAACTTCCCATTGGTATGTAAATTCCAAATGGCAGATGCT
GTCAATAACCTTGCCATGGATGACCTTTGTGTAGGTAGTCCTTGCACCTC
ATGCAGGATAAGCCATTTTAACTTTCTACAATGGGTGCCTCAATAGTTTC
ATAATCTTCATGAAGTTGCATCCTTTGGCAGCTTCTTACAGTTTATTTTC
ACTTCCAATGTAGCAATAAAATAATAAATATAATCGTT

TB16 RNA allele2

(SEQ ID NO:3)

GCGGGAACGCTAACCTGGTCCGGAGCGAGTCTGGGTCTCAGCCCCGCGAA
CAGCCTTTCACGAGTCTTCAAGCTTTCAGGCTATCTTCTAGTCAAGATGA
GTGATAAGCCAGACTTGTCGGAAGTGGAGAAGTTTGACAGGTCAAAACTG
AAGAAAACTAATACTGAAGAAAAAAATACTCTTCCCTCAAAGGAAACTAT
CCAGCAAGAGAAAGAGTGTGTTCAAACATCATAAAATGGGGATCGCCTCC
CAACAGCAGATTTCGACATTACCTGAGAGTCTTGATTTTAGGCTTGTTTT
TTGTAAACCCATGTGTTTGTAGAGATTTTAGGCGTCTTCGGATATCTTCT
CACCTATGTTCCCTGGCTAAGAAGTCAGAGGTAGCCAATGTTTCCTTAAA
TTCATTTTTAAACTTACCATTGGTGCATATGTTCCAGATGGCAGATGCTG
TCAATAATCTCACCATTGATGACCTTTGTGTATGTAGTTCTTGCATCCTA
TACTGGATAAGCCTGTTTTAACCTGCTATGATGGGTGCTTCCATTGCTTC
ATAATCTTCATGAAGTTGCATGCTTTTGCAGCTTTTCACAGTTTATTTGC
ATTTCTAATGTAGTAATAAAGTAACCAATATAATCATT

We claim:

1. A method of diagnosing cancer in a patient, comprising:
a) obtaining a test sample from a patient;
b) measuring the level of thymosin β16 in the test sample; and
c) comparing the level of thymosin β16 in the test sample with the level of thymosin β16 present in a normal control sample;
wherein a higher level of thymosin β16 in the test sample as compared to the level in the normal control sample is indicative of cancer.

2. The method of claim 1, wherein said test sample and said normal control sample are selected from the group consisting of blood, tissue, serum, stool, urine, sputum, cerebrospinal fluid, nipple aspirates, and supernatant from cell lysate.

3. The method of claim 1, wherein the cancer is prostate cancer, lung carcinoma, breast carcinoma, thyroid carcinoma, brain cancers (cerebellum, medulloblastoma, astrocytoma, ependymoma, glioblastoma), pancreatic carcinoma, ovarian carcinoma, eye cancer (retinoblastoma), muscle (rhabdosarcoma), lymphoma, stomach cancer, liver cancer, colon cancer, kidney cancer.

4. A method for prognostic evaluation of a patient suspected of having or having cancer comprising:
a) measuring the level of thymosin β16 in a test sample obtained from a patient;
b) comparing the level determined in step (a) to a range of thymosin β16 known to be present in a biological sample obtained from a normal patient that does not have cancer; and
c) evaluating the prognosis of said patient based on the comparison of step (b), wherein a high level of thymosin β16 in step (a) indicates an aggressive form of cancer and therefore a poor prognosis.

5. The method of claim 4, wherein said test sample is selected from the group consisting of blood, tissue, serum, stool, urine, sputum, cerebrospinal fluid, nipple aspirates, and supernatant from cell lysate.

6. The method of claim 4, wherein the cancer is prostate cancer, lung carcinoma, breast carcinoma, thyroid carcinoma, brain cancers (cerebellum, medulloblastoma, astrocytoma, ependymoma, glioblastoma), pancreatic carcinoma, ovarian carcinoma, eye cancer (retinoblastoma), muscle (rhabdosarcoma), lymphoma, stomach cancer, liver cancer, colon cancer, kidney cancer.

7. The method of claim 1, wherein the level of thymosin β16 is measured by measuring the levels of thymosin β16 mRNA.

8. The method of claim 7, wherein the mRNA is detected by use of an RNA dependent polymerase chain reaction.

9. The method of claim 7, wherein the mRNA is detected by Northern blot analysis by hybridizing mRNA from said test sample or said control sample to a thymosin β16 nucleotide probe.

10. The method of claim 7, wherein the mRNA is detected by DNA microarray analysis.

11. The method of claim 1, wherein the level of thymosin β16 is measured by measuring the levels of thymosin β16 protein.

12. The method of claim 11, wherein thymosin β16 protein level is measured by Mass Spectrometry.

13. The method of claim 11, wherein the method of measuring the level of thymosin β16 levels comprises the steps of:
a) contacting a sample or preparation thereof with an antibody or antibody fragment which selectively binds thymosin β16; and
b) detecting whether said antibody or said antibody fragment is bound by said sample and thereby measuring the levels of thymosin β16 present.

14. The method according to claim 13 wherein said antibody, or said antibody fragment, is detectably labeled.

15. A kit for measuring thymosin β16 levels comprising separate vials containing antibodies, or antibody fragments, which selectively bind human thymosin β16.

16. A kit for measuring thymosin β16 levels comprising at least one polynucleotide sequence that hybridizes to the nucleotide sequence of SEQ ID NO:2 or SEQ ID NO:3.

17. The method of claim 4, wherein the level of thymosin β16 is measured by measuring the levels of thymosin β16 mRNA.

18. The method of claim 17, wherein the mRNA is detected by use of an RNA dependent polymerase chain reaction.

19. The method of claim 17, wherein the mRNA is detected by Northern blot analysis by hybridizing mRNA from said test sample or said control sample to a thymosin β16 nucleotide probe.

20. The method of claim 17, wherein the mRNA is detected by DNA microarray analysis.

21. The method of claim 4, wherein the level of thymosin β16 is measured by measuring the levels of thymosin β16 protein.

22. The method of claim 21, wherein thymosin β16 protein level is measured by Mass Spectrometry.

23. The method of claim 21, wherein the method of measuring the level of thymosin β16 levels comprises the steps of:
a) contacting a sample or preparation thereof with an antibody or antibody fragment which selectively binds thymosin β16; and
b) detecting whether said antibody or said antibody fragment is bound by said sample and thereby measuring the levels of thymosin β16 present.

24. The method according to claim 23 wherein said antibody, or said antibody fragment, is detectably labeled.


SEQUENCE LISTING (3 sequences)

SEQ ID NO: 1 - protein, 45 aa, Homo sapiens
MSDKPDLSEVEKFDRSKLKKTNTEEKNTLPSKETIQQEKECVQTS

SEQ ID NO: 2 - DNA, 639 bp, Homo sapiens
gcgggaaggctaacctggtgcggagccagcctgggtctcagccccgcgtacggcctttcacgagtcttcaagccttcaggctttcttctagtcaagatgagtgataaaccagacttgtcggaagtggagaagtttgacaggtcaaaactgaagaaaactaatactgaagaaaaaaatactcttccctcaaaggaaactatccagcaggagaaagagtgtgttcaaacatcataaaatggggatctcctccaaagagcagatttcagcattgcctgacagtcttggttttaggcttgtttttttgtaaacctgtgtgtttgtagagatttcagacatcttctgatttcttctcacctatattccctggttaagaggtcaggggtagtgaatgtttccttaagttcctttttaaacttcccattggtatgtaaattccaaatggcagatgctgtcaataaccttgccatggatgacctttgtgtaggtagtccttgcacctcatgcaggataagccaattttaactttctacaatgggtgcctcaatagtttcataatcttcatgaagttgcatcctttggcagcttcttacagtttattttcacttccaatgtagcaataaaataataaatataatcgtt

SEQ ID NO: 3 - DNA, 638 bp, Homo sapiens
gcgggaacgctaacctggtccggagcgagtctgggtctcagccccgcgaacagcctttcacgagtcttcaagctttcaggctatcttctagtcaagatgagtgataagccagacttgtcggaagtggagaagtttgacaggtcaaaactgaagaaaactaatactgaagaaaaaaatactcttccctcaaaggaaactatccagcaagagaaagagtgtgttcaaacatcataaaatggggatcgcctcccaacagcagatttcgacattacctgagagtcttgattttaggcttgttttttgtaaacccatgtgtttgtagagattttaggcgtcttcggatatcttctcacctatgttccctggctaagaagtcagaggtagccaatgtttccttaaattcatttttaaacttaccattggtgcatatgttccagatggcagatgctgtcaataatctcaccattgatgacctttgtgtatgtagttcttgcatcctatactggataagcctgttttaacctgctatgatgggtgcttccattgcttcataatcttcatgaagttgcatgcttttgcagcttttcacagtttatttgcatttctaatgtagtaataaagtaaccaatataatcatt